(19) United States
(12) Patent Application Publication
(10) Pub. No.: US 2017/0007457 A1
(43) Pub. Date: Jan. 12, 2017

(54) Anti-Astigmatic Keratotomy

(71) Applicant: Peter Hoffmann, Castrop-Rauxel (DE)

(72) Inventor: Peter Hoffmann, Castrop-Rauxel (DE)

(21) Appl. No.: 15/115,749

(22) PCT Filed: Jan. 30, 2015
(86) PCT No.: PCT/EP2015/051970

§ 371 Date: Aug. 1, 2016

(30) Foreign Application Priority Data
Jan. 30, 2014 (DE) 10 2014 001 081.3

(51) Int. Cl. A61F 9/008

(52) U.S. Cl. CPC A61F 9/00836; A61F 2009/00851; A61F 2009/00853; A61F 2009/00872

(57) ABSTRACT

The invention relates to an ophthalmological instrument for carrying out a laser-surgical intervention on the eye (4), comprising a femtosecond laser (1), which emits pulsed laser radiation (2), an objective (3), from which the laser radiation (2) emerges in a focused manner in the direction of the eye (4), at least one deflection device (6), which deflects the laser radiation (2) and which varies the focal position within the cornea (5) of the eye (4), and a program-controlled control device (8), which is connected to the deflection device (6) and configured to generate a predetermined cut profile within the cornea (5), wherein the cut profile comprises at least one arcuate incision (9) in the periphery of the cornea (5). It is an object of the invention to provide an improved ophthalmological instrument for anti-astigmatic keratotomy with the femtosecond laser (1). This object is achieved by the invention by virtue of the program-controlled control device (8) being furthermore configured to vary the cut depth (D) along the arcuate incision (9), the cut depth (D) reducing toward the ends of the arc. Moreover, the invention relates to a computer program for controlling an ophthalmological instrument.

The invention relates to an ophthalmological instrument for carrying out a laser-surgical intervention on the eye, comprising a femtosecond laser, which emits pulsed laser radiation, an objective, from which the laser radiation emerges in a focused manner in the direction of the eye, at least one deflection device, which deflects the laser radiation and which varies the focal position within the cornea of the eye, and a program-controlled control device, which is connected to the deflection device and configured to generate a predetermined cut profile within the cornea, wherein the cut profile comprises at least one arcuate incision in the periphery of the cornea.

The invention resides in the field of refractive surgery. This is understood to mean interventions on the eye which change the overall refractive power of the eye and thus replace conventional optical corrections such as spectacles or contact lenses, or which should at least significantly reduce the required strength thereof. In refractive surgery, the refractive power is changed by influencing the curvature of the cornea, either by tissue ablation or by defined incisions which, substantially due to the intraocular pressure, bring about a change in form. These days, the tissue ablation for changing the corneal curvature is predominantly carried out by means of a laser.

Femtosecond lasers, i.e. lasers which emit ultrashort light pulses, to be precise predominantly in the infrared wavelength range, have been used for several years now for removing corneal tissue or for generating incisions into the cornea. Using such a laser, a defined cut profile is generated by a suitable program-controlled control device by virtue of small cavitation bubbles being generated in the tissue of the cornea. These cavitation bubbles arise in the focus of the laser, where the necessary energy density is achieved. The unfocused laser beam is hardly absorbed by the corneal tissue, and so a precise cut profile can be generated at any depth within the cornea. Suitable ophthalmological instruments, which enable three-dimensional incision by a program-controlled guidance of the focus of the laser beam in all three spatial directions (x, y, z) are known from the prior art; see e.g. WO 2010/142311 A1.

The methods of refractive surgery also include anti-astigmatic keratotomy. Strong astigmatism can be corrected by the anti-astigmatic keratotomy. The conditions are equalized by arcuate relief cuts in the periphery of the cornea and the astigmatism is corrected. This method is very effective but it is difficult to meter.

In recent times, it has been suggested to carry out the anti-astigmatic keratotomy using the femtosecond laser, particularly in the case of (strong) astigmatism after a corneal transplant. This program-controlled method is potentially more precise than the conventional method which is carried out manually by means of a depth-calibrated diamond knife. However, it was found that the predictability and metering is only mediocre, even when using the femtosecond laser for correcting astigmatism. A principal problem of the method is that the geometry of the cornea is modified by the arcuate incisions with a predetermined cut depth in such a way that, even if the astigmatism overall is reduced, aberrations of higher order are induced, and these may be perceivable by the patient. The reason for this is that steps in the tension distribution of the cornea are generated at the ends of the arcuate incisions, said steps locally influencing the refractive power. This is not remedied either by the use of the femtosecond laser with a precise and program-controlled guidance of the focus.

Against this backdrop, it is an object of the invention to provide an improved ophthalmological instrument for the anti-astigmatic keratotomy with the femtosecond laser.

Proceeding from an instrument of the type set forth at the outset, the invention solves this object by virtue of the program-controlled device being furthermore configured to vary the cut depth along the arcuate incision, the cut depth reducing toward the ends of the arc.

The invention is based on the discovery that the use of the femtosecond laser renders it possible to vary the cut depth along the arcuate incision in a targeted and precise manner. In other words, this means that the incision introduced into the cornea has different depths at different positions. If the arcuate cut is circular-arc-shaped, it is possible, for example, to specify the cut depth as a function of the arc angle.

In accordance with the invention, the introduced incision can level off gently toward the ends of the arc and, as a result thereof, be embodied in such a way that the above-described steps in the tension distribution are reduced or even completely avoided. As a result of using the ophthalmological instrument according to the invention, the aforementioned unwanted side effects of anti-astigmatic keratotomy (aberrations of higher order) are avoided or reduced; the predictability is improved.

In practice, the program-controlled control device of the ophthalmological instrument according to the invention is programmed in such a way that the cut depth varies e.g. between 0% and 80% of the corneal thickness along the arcuate incision, depending on the strength of the astigmatism. The minimum cut depth and the maximum cut depth are variable. By varying the maximum cut depth, the effect, obtained according to the invention, for correcting the astigmatism can be metered. Here, it is possible, in order to obtain an effect which is as uniform as possible, for the cut depth to the greatest in the center of the arc, while it reduces toward the ends of the arc. Approximately the upper 60 μm of the cornea are immediately re-growing epithelium. Therefore, it may be expedient to set the minimum cut depth to be 60 μm.

By way of example, the cut depth at the ends of the arc can correspond to less than 50%, preferably less than 30%, particularly preferably less than 10% of the maximum cut depth. In the ideal case, the depth of the incision is reduced toward zero toward the ends of the arc. Hence, the incisions level off gently toward the ends of the arc, and the effects on the deformation of the cornea are less irregular than in the case of conventional methods.

In a preferred embodiment of the invention, the ophthalmological instrument has a device for optical coherence tomography, which carries out an accurate, high-resolution measurement of the cornea during the operation. Then, the program-controlled control device can advantageously be configured to determine the cut depth according to the stipulation of an absolute or relative distance (e.g. relative to the corneal thickness), which varies along the arcuate incision, from the corneal inner layer, the distance being established by means of the device for optical coherence tomography. The corresponding control of the cut depth is readily implementable during the intervention by means of optical coherence tomography. As a result, a possible transfer error from the pre-operative measurement to the intraoperative implementation is dispensed with.

In a preferred embodiment of the ophthalmological instrument according to the invention, the cut depth of the arcuate incision is determined by a polynomial or any other mathematical function (e.g. also a sinusoidal function), which specifies the cut depth as a function of the arc angle. In the simplest case, the function can be a parabolic function, wherein the vertex of the parabola marks the deepest point of the incision, which can lie approximately in the arc center, as explained above. The deepest point of the incision can also lie away from the arc center, for example if an asymmetric astigmatism is intended to be corrected.

In a preferred embodiment of the invention, the program-controlled control device of the ophthalmological instrument is configured to generate two circular-arc-shaped incisions, which lie symmetrically opposite one another in relation to the corneal center, with an arc length of substantially 90°. This type of incision was found to be effective for correcting astigmatism.

The invention furthermore relates to a computer program for controlling an ophthalmological instrument, in particular of the type described above, with instructions for generating a predetermined cut profile within the cornea, wherein the cut profile comprises at least one arcuate incision in the periphery of the cornea. According to the invention, the computer program has instructions for varying the cut depth along the arcuate incision. According to the invention, a conventional ophthalmological instrument with the femtosecond laser already in clinical use today can be retrofitted by virtue of the computer program being installed on the program-controlled control device for guidance of the focal position. The computer program can be made available on a known type of data medium, or else it can be made available for download from the Internet.

Exemplary embodiments of the invention are explained below on the basis of the drawings. In detail:

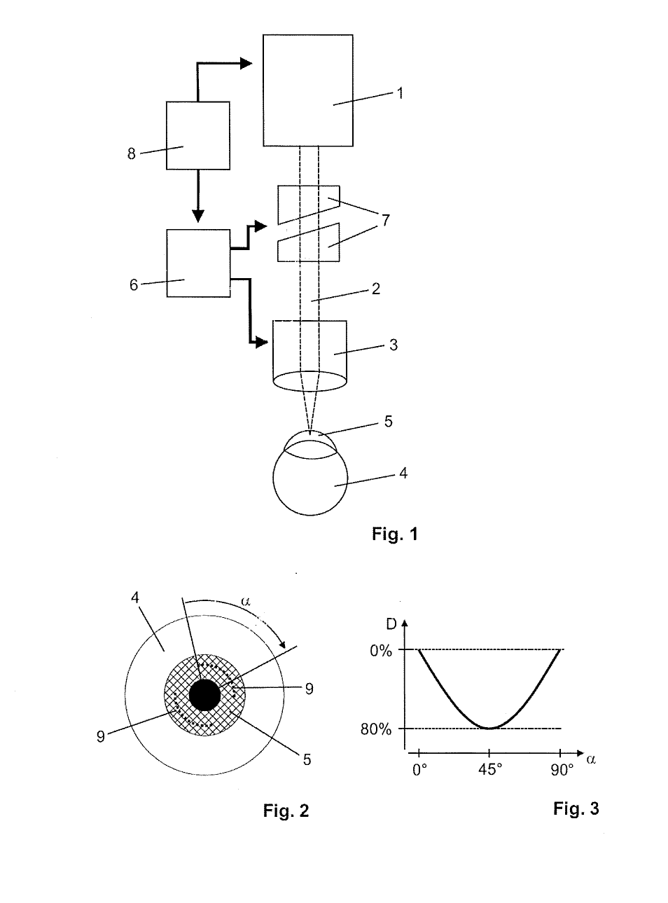

FIG. 1 shows an ophthalmological instrument according to the invention in a block diagram;

FIG. 2 shows a schematic plan view of an eye with arcuate incisions for correcting an astigmatism;

FIG. 3 shows an illustration of the incision according to the invention on the basis of a diagram.

FIG. 1 shows an ophthalmological instrument according to the invention as a block diagram. The instrument comprises a femtosecond laser 1 which emits pulsed laser radiation 2. The laser radiation 2 is directed in the direction of an eye 4 to be treated by means of an objective 3. The objective 3 focuses the laser radiation 2 in such a way that the focal position lies within the cornea 5.

Here, provision is made of a deflection device 6, which interacts with an optical assembly 7 in order to vary the beam position in a plane perpendicular to the direction of the laser beam 2 (i.e. in the x- and y-direction). Moreover, the deflection device 6 has a functional connection to the objective 3 for varying the focal length of the objective 3. Overall, it is thus possible to vary the focal position in three dimensions, i.e. in the x-, y- and z-direction. The deflection device 6 is connected to a program-controlled control device 8. The program-controlled control device actuates the deflection device 6 in order to vary the focal position. Here, the program-controlled control device 8 is configured to generate a predetermined cut profile within the cornea 5, wherein the cut profile comprises at least one arcuate incision in the periphery of the cornea. The program-controlled control device 8 also actuates the femtosecond laser 1 in order to respectively switch the latter on and off for the generation of the incisions.

The incision for anti-astigmatic keratotomy in accordance with the invention is shown schematically in FIG. 2. The program-controlled control device 8 is configured to generate two arcuate incisions 9, which lie symmetrically opposite one another in relation to the corneal center. In the depicted exemplary embodiment, the incisions 9 have a circular-arc-shaped form. The arc length is substantially 90° in each case.

FIG. 3 illustrates the variation according to the invention in the cut depth D (in the z-direction), in each case along the arcuate incision 9. The program-controlled control device 8 is configured not only to predetermine the incision for generating the circular arc in accordance with FIG. 2 but also to vary the cut depth D along the respective arcuate incision. The diagram in FIG. 3 shows the cut depth D as a function of the arc angle α. On the basis of the diagram, it is possible to identify that the cut depth D is varied between 0% and 80% of the corneal thickness. The maximum value of the cut depth can vary and depends, in general, on the strength of the astigmatism to be corrected. In the exemplary embodiment, the cut depth is greatest at the arc center and reduces to 0% toward the ends of the arc. In the depicted exemplary embodiment, the cut depth is defined by a parabola. The parabola specifies the cut depth D as a function of the arc angle α. The vertex of the parabola lies at an arc angle of 45°, i.e. in the center of the arcuate incision.

As a result of the leveling-off of the arcuate incision towards the edges at a =0° and a =90°, shown in FIG. 3, the higher-order aberrations occurring in conventional anti-astigmatic keratotomy are avoided or in any case reduced. As a result, it becomes easier to meter and better predict the method.

1. An ophthalmological instrument for carrying out a laser-surgical intervention on the eye (4), comprising a femtosecond laser (1), which emits pulsed laser radiation (2), an objective (3), from which the laser radiation (2) emerges in a focused manner in the direction of the eye (4), at least one deflection device (6), which deflects the laser radiation (2) and which varies the focal position within the cornea (5) of the eye (4), and a program-controlled control device (8), which is connected to the deflection device (6) and configured to generate a predetermined cut profile within the cornea (5), wherein the cut profile comprises at least one arcuate incision (9) in the periphery of the cornea (5), wherein the program-controlled device (8) is furthermore configured to vary the cut depth (D) along the arcuate incision (9), the cut depth (D) reducing toward the ends of the arc.

2. The ophthalmological instrument as claimed in claim 1, wherein the program-controlled control device (8) is configured to vary the cut depth (D) between 0% and 80% of the corneal thickness.

3. The ophthalmological instrument as claimed in either of claim 1, wherein the cut depth (D) at the ends of the arc corresponds to less than 50%, preferably less than 30%, particularly preferably less than 10% of the maximum cut depth.

4. The ophthalmological instrument as claimed in claim 1, wherein the cut depth (D) is determined by a polynomial or any other mathematical function, which specifies the cut depth (D) as a function of the arc angle (α).

5. The ophthalmological instrument as claimed in claim 4, wherein the polynomial is a parabolic function.

6. The ophthalmological instrument as claimed in claim 4, wherein the mathematical function is a sinusoidal function.

7. The ophthalmological instrument as claimed in claim 1, wherein the program-controlled control device (8) is configured to generate two circular-arc-shaped incisions (9), which lie symmetrically opposite one another in relation to the corneal center, with an arc length of substantially 90°.

8. The ophthalmological instrument as claimed in claim 1, further comprising a device for optical coherence tomography.

9. The ophthalmological instrument as claimed in claim 8, wherein the program-controlled control device (8) is configured to determine the cut depth (D) according to the stipulation of an absolute or relative distance, which varies along the arcuate incision (9), from the corneal inner layer, the distance being established by means of the device for optical coherence tomography.

10. A computer program for controlling an ophthalmological instrument, with instructions for generating a predetermined cut profile within the cornea (5) of an eye (4), wherein the cut profile comprises at least one arcuate incision (9) in the periphery of the cornea (5), further comprising instructions for varying the cut depth (D) along the arcuate incision (9), the cut depth (D) reducing toward the ends of the arc.
